(19) United States
(12) Patent Application Publication
(10) Pub. No.: US 2011/0237773 A1
(43) Pub. Date: Sep. 29, 2011

(54) Glycerol treatment process

(75) Inventor: Patrick Gilbeau, Braine-le-Comte (BE)

(73) Assignee: SOLVAY SA, Brussels (BE)

(21) Appl. No.: 13/131,516

(22) PCT Filed: Dec. 7, 2009
(86) PCT No.: PCT/EP2009/066478

§ 371 Date: May 26, 2011

(30) Foreign Application Priority Data
Dec. 8, 2008 (FR) 08.58362

(51) Int. Cl. C08G 65/24; C07C 29/88; C07D 301/02; C07C 41/02

(52) U.S. Cl. 528/421; 568/852; 549/514; 568/579

(57) ABSTRACT

Process for treating a glycerol product contaminated with at least one glycerol alkyl ether in order to convert at least one portion of the glycerol alkyl ether to glycerol, in which the glycerol product is subjected to a reaction with at least one halo-de-alkoxylation agent introduced during the treatment, and in which the molar ratio of the total amount of the halo-de-alkoxylation agent to the total amount of the glycerol alkyl ether present in the glycerol product before the treatment, is greater than or equal to 0.1 and less than or equal to 1 000 000.

The present patent application claims the benefit of the following patent application FR 0858362 filed on 8 Dec. 2008, the content of which is incorporated here by reference.

The present invention relates to a glycerol treatment process. The present invention relates more specifically to a process for treating glycerol contaminated with glycerol alkyl ethers.

Glycerol may be used in various applications, especially in the manufacture of dichloropropanol, a reaction intermediate in the manufacture of epichlorohydrin and epoxy resins (Kirk-Othmer Encyclopedia of Chemical Technology, Fourth Edition, 1992, Vol. 2, page 156, John Wiley & Sons, Inc.).

International Application WO 2007/144335 filed in the name of SOLVAY S A discloses a process for manufacturing dichloropropanol via chlorination of glycerol, in which glycerol is first purified of glycerol alkyl ethers. The purification treatments disclosed are evaporative concentration, evaporative crystallization, distillation, fractional distillation, stripping or liquid/liquid extraction treatments. These treatments complicate the dichloropropanol manufacturing process.

The invention aims to solve this problem by providing a process for treating a glycerol product contaminated with at least one glycerol alkyl ether in order to convert at least one portion of the glycerol alkyl ether to glycerol, in which the glycerol product is subjected to a reaction with at least one halo-de-alkoxylation agent, and in which the molar ratio of the total amount of the halo-de-alkoxylation agent introduced during the treatment to the total amount of the glycerol alkyl ether present in the glycerol product before the treatment, is greater than or equal to 0.1 and less than or equal to 1 000 000.

In the process for treating a contaminated glycerol product according to the invention, the treatment generally leads, in addition, to the formation of at least one compound that contains the alkyl group of the glycerol alkyl ether and that is neither a glycerol alkyl ether nor an alkyl ether of a glycerol halohydrin. This compound generally has a lower boiling point than the glycerol alkyl ether from which it is obtained.

One of the essential features of the present invention lies in the conversion of glycerol alkyl ethers to glycerol and possibly to glycerol halohydrin.

Compared to the removal of glycerol alkyl ethers by the processes disclosed in International Application WO 2007/144335, the process according to the invention has the following advantages:
a) the recovery of glycerol that can be reused following the halo-de-alkoxylation reaction;
b) an easier separation of the compound containing the alkyl group, from the reaction medium, as a result of the greater volatility of this compound compared to that of the glycerol alkyl ethers.

It has surprisingly been found that the glycerol alkyl ethers present in a contaminating amount in the glycerol product can be converted into glycerol while the glycerol present in the contaminated glycerol product before the treatment or formed during the treatment remains largely unaffected by the treatment with the halo-de-alkoxylation agent.

In the remainder of the document the expression “glycerol product contaminated with at least one glycerol alkyl ether” will be denoted by “contaminated glycerol product”.

In the process for treating contaminated glycerol product according to the invention, the glycerol content of the contaminated glycerol product before the treatment is generally greater than or equal to 200 g/kg, often greater than or equal to 500 g/kg, frequently greater than or equal to 750 g/kg, routinely greater than or equal to 900 g/kg, specifically greater than or equal to 950 g/kg, particularly greater than or equal to 990 g/kg, specifically greater than or equal to 995 g/kg and in particular greater than or equal to 999 g/kg.

In the process for treating contaminated glycerol product according to the invention, the glycerol alkyl ether may be chosen from the group consisting of glycerol alkyl monoethers, glycerol alkyl diethers, glycerol alkyl triethers, and any mixture of at least two of them. In these glycerol alkyl ethers, the alkyl groups are generally selected independently from the group consisting of methyl, ethyl, propyl, butyl, pentyl, hexyl, heptyl, octyl groups and any combination of at least two of them. These glycerol alkyl ethers are as described in Application WO 2007/144335 filed in the name of Solvay S A, of which the content, and more specifically the passage from page 2, line 6 to page 3, line 25, is incorporated herein by reference.

Glycerol oligomers are not considered to be glycerol alkyl ethers.

In the process for treating contaminated glycerol product according to the invention, the glycerol alkyl ether is preferably chosen from the group consisting of glycerol methyl monoethers, glycerol methyl diethers, glycerol methyl triethers, and any mixture of at least two of them. The glycerol methyl monoethers, namely 3-methoxy-1,2-propanediol and 2-methoxy-1,3-propanediol, and mixtures thereof, are more preferred.

In the process for treating contaminated glycerol product according to the invention, the glycerol alkyl ether content in the contaminated glycerol product before the treatment is generally greater than or equal to 0.001 g/kg of contaminated glycerol product, often greater than or equal to 0.005 g/kg, frequently greater than or equal to 0.01 g/kg, routinely greater than or equal to 0.04 g/kg and usually greater than or equal to 0.1 g/kg. This content is generally less than or equal to 100 g/kg, often less than or equal to 90 g/kg, routinely less than or equal to 50 g/kg, frequently less than or equal to 10 g/kg, usually less than or equal to 1 g/kg, commonly less than or equal to 0.5 g/kg and particularly less than or equal to 0.2 g/kg.

In the process for treating contaminated glycerol product according to the invention, the glycerol alkyl ether is preferably a glycerol methyl ether as defined above, and the content of glycerol methyl ethers in the contaminated glycerol product before the treatment is generally greater than or equal to 0.001 g/kg of contaminated glycerol product, often greater than or equal to 0.005 g/kg, frequently greater than or equal to 0.01 g/kg, routinely greater than or equal to 0.04 g/kg and usually greater than or equal to 0.1 g/kg. This amount is generally less than or equal to 100 g/kg, often less than or equal to 90 g/kg, routinely less than or equal to 50 g/kg, frequently less than or equal to 10 g/kg, usually less than or equal to 1 g/kg, commonly less than or equal to 0.5 g/kg and particularly less than or equal to 0.2 g/kg.

In the process for treating contaminated glycerol product according to the invention, the glycerol content in the contaminated glycerol product before the treatment is preferably greater than or equal to 900 g/kg of contaminated glycerol product, more preferably greater than or equal to 950 g/kg, particularly preferably greater than or equal to 990 g/kg, and the content of glycerol methyl ethers in the contaminated glycerol product before the treatment is as described above.

In the process for treating contaminated glycerol product according to the invention, the contaminated glycerol product may also contain at least one diol as described in Application WO 2009/000773 in the name of Solvay S A, from page 2, line 11 to page 3, line 21, the content of which is incorporated herein by reference.

The diol is preferably chosen from the group consisting of 1,2-ethanediol (ethylene glycol), 1,2-propanediol (propylene glycol), 1,3-propanediol, 1,2-butanediol, 2,3-butanediol, 1,4-butanediol and any mixture of at least two of them. 1,3-propanediol is often present.

The diol content in the contaminated glycerol product before the treatment is generally greater than or equal to 0.001 g diol/kg of contaminated glycerol product, often greater than or equal to 0.005 g/kg, frequently greater than or equal to 0.01 g/kg, routinely greater than or equal to 0.04 g/kg and usually greater than or equal to 0.1 g/kg. This content is generally less than or equal to 100 g diol/kg, often less than or equal to 90 g/kg, routinely less than or equal to 50 g/kg, frequently less than or equal to 10 g/kg, usually less than or equal to 1 g/kg, commonly less than or equal to 0.5 g/kg and particularly less than or equal to 0.2 g/kg.

In the process for treating contaminated glycerol product according to the invention, the contaminated glycerol product may also contain at least one monoalcohol as described in International Application WO 2007/144335 in the name of Solvay S A, on page 3, lines 26 to 31, the content of which is incorporated herein by reference.

The monoalcohol content in the contaminated glycerol product before the treatment is usually greater than or equal to 0.001 g/kg of contaminated glycerol product, and often greater than or equal to 0.01 g/kg. This content is generally less than 20 g/kg of contaminated glycerol product and often less than or equal to 2 g/kg.

In the process for treating contaminated glycerol product according to the invention, the contaminated glycerol product may also contain water.

The water content in the contaminated glycerol product before the treatment is generally greater than or equal to 0.1 g/kg of contaminated glycerol product. This content is generally less than or equal to 100 g/kg, often less than or equal to 50 g/kg, frequently less than or equal to 20 g/kg, specifically less than or equal to 100 g/kg and particularly less than or equal to 1 g/kg.

In the process for treating contaminated glycerol product according to the invention, the contaminated glycerol product may also contain at least one compound chosen from the group consisting of alkyl esters, e.g. alkyl esters of fatty acids, fatty acids, glycerol esters, salts, and mixtures of at least two of these compounds, as described in Application WO 2007/144335 in the name of Solvay S A, on page 5, lines 12 to 20, the content of which is incorporated herein by reference.

The content of alkyl esters, e.g. alkyl esters of fatty acids, in the contaminated glycerol product before the treatment is generally greater than or equal to 0.1 g/kg of contaminated glycerol product, often greater than or equal to 1 g/kg and frequently greater than or equal to 5 g/kg. This content is generally less than 50 g/kg of contaminated glycerol product, and often less than or equal to 30 g/kg and more often less than or equal to 10 g/kg.

The content of glycerol esters in the contaminated glycerol product before the treatment is generally greater than or equal to 0.1 g/kg of contaminated glycerol product, often greater than or equal to 1 g/kg and frequently greater than or equal to 5 g/kg. This content is generally less than 50 g/kg of contaminated glycerol product, and often less than or equal to 30 g/kg and more often less than or equal to 10 g/kg.

The salt content in the contaminated glycerol product before the treatment is generally greater than or equal to 0.0005 g/kg of contaminated glycerol product, often greater than or equal to 0.001 g/kg and frequently greater than or equal to 0.01 g/kg. This content is generally less than 10 g/kg, and often less than or equal to 1 g/kg and more often less than or equal to 0.1 g/kg.

The fatty acid content in the contaminated glycerol product before the treatment is generally greater than or equal to 0.001 g/kg of contaminated glycerol product, often greater than or equal to 0.01 g/kg and frequently greater than or equal to 0.05 g/kg. This content is generally less than 10 g/kg, and often less than or equal to 5 g/kg and more often less than or equal to 1 g/kg.

In the process for treating contaminated glycerol product according to the invention, the contaminated glycerol product may also contain at least one nitrogen-containing compound as described in Application WO 2009/077528 in the name of Solvay S A, on page 2, lines 22 to page 3, line 17, the content of which is incorporated herein by reference.

The total content of nitrogen-containing compound in the contaminated glycerol product before the treatment expressed as elemental nitrogen is usually less than or equal to 1 g of N/kg of contaminated glycerol product, often less than or equal to 0.5 g N/kg, frequently less than or equal to 0.1 g N/kg, routinely less than or equal to 0.05 g N/kg, specifically less than or equal to 0.03 g N/kg, and particularly less than or equal to 0.01 g N/kg. This content is generally greater than or equal to 0.1 mg N/kg.

In the process for treating contaminated glycerol product according to the invention, the contaminated glycerol product may also contain at least one glycerol oligomer as described in Application WO 2009/121853 in the name of Solvay S A, the content of which is incorporated herein by reference, more specifically the passage from page 3, line 2 to page 6, line 19.

The glycerol oligomer is preferably a glycerol dimer chosen from the group consisting of glycerol dimers of linear structure, glycerol dimers of branched structure, glycerol dimers of cyclic structure, and any mixture of at least two of them.

In the process for treating contaminated glycerol product according to the invention, the content of the cyclic oligomer of glycerol, preferably cyclic dimer of glycerol, is often less than or equal to 10 g of cyclic oligomer/kg of contaminated glycerol product, frequently less than or equal to 5 g/kg, commonly less than or equal to 2.5 g/kg, specifically less than or equal to 1 g/kg, more specifically less than or equal to 0.5 g/kg and particularly less than or equal to 0.1 g/kg. This content is often greater than or equal to 0.05 g/kg.

The diols, glycerol alkyl ethers, monoalcohols, water, alkyl esters, e.g. alkyl esters of fatty acids, fatty acids, glycerol esters, salts, nitrogen containing compounds and glycerol oligomers may be by-products of glycerol manufacturing processes such as, for example, the processes for conversion of oils and/or fats of animal and/or plant origin via transesterification, and/or saponification and/or hydrolysis and/or ammonolysis reactions.

In the process for treating contaminated glycerol product according to the invention, the halo-de-alkoxylation agent may be used in any form selected from the group consisting of solid, liquid, gas, solution, dispersion, emulsion, suspension, and any combination of at least two of these forms.

In the process for treating contaminated glycerol product according to the invention, at least one portion of the halo-de-alkoxylation agent is often used in gas form. In the process for treating contaminated glycerol product according to the invention, at least one portion of the halo-de-alkoxylation agent is often supplied in gas form during the reaction.

The portion of the halo-de-alkoxylation agent used or supplied in the gas form is usually at least 50% mol, preferably at least 75% mol, more preferably at least 90% mol, yet more preferably at least 95% mol, still more preferably at least 99% mol and most preferably at least 99.9% mol of the total amount of the halo-de-alkoxylation agent used or supplied during the treatment.

A halo-de-alkoxylation agent used or supplied essentially in the gas form is convenient.

When in gas form, the halo-de-alkoxylation agent may be used optionally in admixture with at least one other gaseous compound. The other gaseous compound may be selected from the group consisting of nitrogen, oxygen, carbon dioxide, steam, a noble gas, and any mixture of at least two of them. The content of the halo-de-alkoxylation agent in the mixture is usually greater than or equal to 50% mol, often greater than or equal to 80% mol, frequently greater than or equal to 90% mol and more specifically greater than or equal to 99% mol. A gas mixture consisting essentially of the halo-de-alkoxylation agent is particularly convenient.

In the process for treating contaminated glycerol product according to the invention, the halo-de-alkoxylation agent may contain at least one of hydrogen halides, Lewis acids, and halotrialkylsilanes. Hydrogen halides are particularly suitable. Mixtures of at least two hydrogen halides may also be suitable.

In the process for treating contaminated glycerol product according to the invention, the halo-de-alkoxylation agent may contain at least one hydrogen halide.

The hydrogen halide may be chosen from the group consisting of hydrogen fluoride, hydrogen chloride, hydrogen bromide, hydrogen iodide, and any mixture of at least two of them.

The hydrogen halide may be in the form of a gas or of an aqueous solution or of a solution in a non-aqueous solvent, or of a combination of at least two of these forms.

When the hydrogen halide is in the form of an aqueous solution, the amount of hydrogen halide relative to the sum of the amounts of hydrogen halide and of water is usually less than or equal to 70% by weight, often less than or equal to 50% by weight, frequently less than or equal to 40% by weight, particularly less than or equal to 25% by weight and specifically less than or equal to 10% by weight. This amount is usually greater than or equal to 1% by weight.

When the hydrogen halide is in the form of an aqueous solution, it may also be advantageous that the amount of hydrogen halide relative to the sum of the amounts of hydrogen halide and of water is higher than or equal to 40% by weight and preferably higher than or equal to 50% by weight.

Hydrogen chloride is one hydrogen halide that is very suitable.

In the process for treating contaminated glycerol product according to the invention, the hydrogen halide may contain hydrogen chloride.

The hydrogen chloride is usually used in the form of a gas, often in the form of an aqueous solution and frequently in the form of a mixture of gas and of an aqueous solution.

The use of gaseous hydrogen chloride is particularly suitable.

When in gas form, the hydrogen chloride may be used optionally in admixture with at least one other gaseous compound. The other gaseous compound may be selected from the group consisting of nitrogen, oxygen, carbon dioxide, steam, a noble gas, and any mixture of at least two of them. The content of the hydrogen chloride in the mixture is usually greater than or equal to 50% mol, generally greater than or equal to 80% mol, in many cases greater than or equal to 90% mol, often greater than or equal to 99% mol, frequently greater than or equal to 99.5% mol and more specifically greater than or equal to 99.9% mol. A gas mixture consisting essentially of hydrogen chloride is particularly convenient.

The use of gaseous halo-de-alkoxylation agent has the following advantages over the use of an aqueous solution of the same agent:
(a) a higher conversation rate of the glycerol alkyl ethers
(b) a lower quantity of water to be separated from the glycerol at the issue of the treatment
(c) the formation of an alkyl chloride co-product easier to separate from the glycerol than the corresponding alkyl alcohol.

All these advantages are particularly encountered when the halo-de-alkoxylation agent is hydrogen chloride.

The Lewis acid may be chosen from the group consisting of boron halides, aluminium halides and any mixture of at least two of them. The halides are often selected from the group consisting of BF₃, BCl₃, (CH₃)₂BBr, BBr₃, BI₃, AlCl₃ and any mixture of at least two of them.

The halotrialkylsilane is frequently iodotrimethylsilane.

In the process for treating contaminated glycerol product according to the invention, the formed compound containing the alkyl group of the glycerol alkyl ether may be chosen from the group consisting of alkyl halides, alcohols, alcoholates, and any mixture of at least two of them.

The formed compound containing the alkyl group of the glycerol alkyl ether is usually an alkyl halide, sometimes an alcohol, and frequently a mixture of the two.

The formed compound containing the alkyl group of the glycerol alkyl ether is preferably an alcohol, more preferably methanol.

The formed compound containing the alkyl group of the glycerol alkyl ether is, in an equally preferred manner, an alkyl chloride, more particularly methyl chloride.

In the process for treating contaminated glycerol product according to the invention, at least one portion of the glycerol alkyl ether may be converted to glycerol halohydrin.

The glycerol halohydrin may be a glycerol monohalohydrin or a glycerol dihalohydrin or a mixture thereof. The glycerol halohydrin is preferably a glycerol monohalohydrin.

The glycerol halohydrin may be chosen from the group consisting of glycerol fluorohydrins, glycerol chlorohydrins, glycerol bromohydrins, glycerol iodohydrins and any mixture of at least two of them. Glycerol chlorohydrins are preferred. Glycerol monochlorohydrin is particularly preferred.

In the process for treating contaminated glycerol product according to the invention, the treatment of the contaminated glycerol product may be carried out in batch mode, in semi-continuous mode or in continuous mode.

The expression “continuous mode” is understood to mean an operating mode in which the reactants, that is to say glycerol containing at least one glycerol alkyl ether and the halo-de-alkoxylation agent continuously feed a reaction medium, and where at least one of the reaction products, that is to say a compound containing the alkyl group of the glycerol alkyl ether or the treated glycerol are continuously withdrawn from said reaction medium.

The expression “batch mode” is understood to mean any other operating mode, encompassing the semi-continuous or fed-batch mode.

In the process for treating contaminated glycerol product according to the invention, the treatment is performed at a temperature generally greater than or equal to 20° C., often greater than or equal to 40° C., frequently greater than or equal to 60° C. and particularly greater than or equal to 75° C. This temperature is usually less than or equal to 160° C., often less than or equal to 140° C., frequently less than or equal to 120° C. and particularly less than or equal to 100° C.

In the process for treating contaminated glycerol product according to the invention, the treatment is performed at a pressure generally greater than or equal to 0.3 bar absolute, often greater than or equal to 0.5 bar absolute, frequently greater than or equal to 0.7 bar absolute and in particular greater than or equal to 0.9 bar absolute. This pressure is generally less than or equal to 100 bar absolute, often less than or equal to 50 bar absolute, frequently less than or equal to 20 bar absolute, in a lot of cases less than or equal to 10 bar absolute and in particular less than or equal to 5 bar absolute.

In the process for treating contaminated glycerol product according to the invention and in the case where the halo-de-alkoxylation agent comprises hydrogen chloride, the treatment is performed at a partial pressure of hydrogen chloride generally greater than or equal to 0.3 bar, often greater than or equal to 0.5 bar, frequently greater than or equal to 0.7 bar and in particular greater than or equal to 0.9 bar. This pressure is generally less than or equal to 100 bar, often less than or equal to 50 bar, frequently less than or equal to 20 bar, in a lot of cases less than or equal to 10 bar and in particular less than or equal to 5 bar.

In the process for treating contaminated glycerol product according to the invention, when the treatment is carried out in batch mode, the treatment time is generally greater than or equal to 10 min, often greater than or equal to 20 min, frequently greater than or equal to 30 min and in particular greater than or equal to 1 h. This time is generally less than or equal to 100 h, often less than or equal to 50 h, frequently less than or equal to 30 h, in a lot of cases less than or equal to 20 h, in particular less than or equal to 10 h and more specifically less than or equal to 5 h.

In the process for treating contaminated glycerol product according to the invention, when the treatment is carried out in continuous mode, the residence time of the treatment, defined as the ratio of the volume of the reaction medium to the sum of the feed flow rates of the reactants, is generally greater than or equal to 10 min, often greater than or equal to 20 min, frequently greater than or equal to 30 min and in particular greater than or equal to 1 h. This residence time is generally less than or equal to 100 h, often less than or equal to 50 h, frequently less than or equal to 30 h, in a lot of cases less than or equal to 20 h, in particular less than or equal to 10 h and more specifically less than or equal to 5 h.

In the process for treating contaminated glycerol product according to the invention, the molar ratio of the total amount of halo-de-alkoxylation agent introduced during the treatment to the total amount of the glycerol alkyl ether present in the contaminated glycerol product before the treatment, is often greater than or equal to 1, frequently greater than or equal to 10, in a lot of cases greater than or equal to 50 and in particular greater than or equal to 100. This ratio is often less than or equal to 100 000, frequently less than or equal to 10 000, in a lot of cases less than or equal to 1000 and in particular less than or equal to 500.

In the process for treating contaminated glycerol product according to the invention, the reaction between the contaminated glycerol product and the halo-de-alkoxylation agent may be carried out in the presence of at least one catalyst for the halogenation of polyhydroxylated alkanes. This catalyst may be a carboxylic acid and/or a carboxylic acid derivative. The carboxylic acid derivative may be chosen from the group consisting of carboxylic acid esters, carboxylic acid chlorides, carboxylic acid anhydrides, carboxylic acid salts, carboxylic acid amides, nitriles, and any mixture of at least two of them, some of which are described in Application WO 2005/054167, in the name of Solvay S A, from page 6, line 28 to page 7, line 35, the content of which is incorporated here by reference, and in Application WO 2006/020234, of which the content, and more specifically the passage from page 12, line 20 to page 18, line 3, is incorporated herein by reference, and in Application WO 2009/077528, in the name of Solvay S A, of which the content, and more specifically the passage on page 10, lines 4 to 6, is incorporated herein by reference.

The catalyst may also be a heteropolyacid such as H₃PMO₁₂₋ₓWₓO₄₀ (x being an integer between 1 and 12, or zero), H₄SiMo₁₂₋ₓWₓO₄₀ (x being an integer between 1 and 12, or zero), H₃₊ₓPW₁₂₋ₓVₓO₄₀ (x being an integer between 1 and 3, or zero) and H₃₊ₓPMo₁₂₋ₓVₓO₄₀ as described by Sang Hee Lee et al. in Catalysis Communications 9, 1920-1923 (2008). The catalyst may also be a metal oxide, a mixed metal oxide, a halide of a metal from groups IIB, IIIB e.g. Sc), IVB (e.g. Ti), VB (e.g. V), IIIA (e.g. Al), IVA (e.g. Sn) and VA (e.g. Bi) of the Periodic Table of the Elements, from group VIII of the Periodic Table of the Elements, such as Fe, Co, Ni, Pd and Pt, from group IIB of the Periodic Table of the Elements, such as Zn, and also a compound chosen from lanthanoids and actinoids, zeolites, heteropolyacid salts, oxo acids or oxo acid salts such as BPO₄, AlPO₄, polyphosphoric acid, phosphoric acid and salts thereof, boric acid and salts thereof, niobic acid or else a strongly acidic organic compound such as a sulphonic acid optionally in the form of an ion-exchange resin as described in Patent JP 2008/214290.

In the process for treating contaminated glycerol product according to the invention, the reaction between the contaminated glycerol product and the halo-de-alkoxylation agent is often carried out in the absence of catalyst.

In one particular embodiment of the process for treating contaminated glycerol product according to the invention, the halo-de-alkoxylation agent comprises gaseous hydrogen chloride, the treatment is carried out under at least one of the following conditions: in continuous mode, at a temperature greater than or equal to 70° C. and less than or equal to 90° C., at a pressure greater than or equal to 0.9 bar absolute and less than or equal to 5 bar absolute, for a residence time greater than or equal to 10 min and less than or equal to 10 h, and for a molar ratio of the total amount of hydrogen chloride introduced during the treatment to the total amount of the glycerol alkyl ether present in the contaminated glycerol product before the treatment, greater than or equal to 10 and less than or equal to 10 000.

In one more particular embodiment of the process for treating contaminated glycerol product according to the invention, the halo-de-alkoxylation agent consists essentially of gaseous hydrogen chloride, and the treatment is carried out under at least one of the following conditions: in continuous mode, at a temperature greater than or equal to 70° C. and less than or equal to 90° C., at a pressure greater than or equal to 0.9 bar absolute and less than or equal to 5 bar absolute, for a residence time greater than or equal to 10 min and less than or equal to 10 h, and for a molar ratio of the total amount of hydrogen chloride introduced during the treatment to the total amount of the glycerol alkyl ether present in the contaminated glycerol product before the treatment, greater than or equal to 10 and less than or equal to 10 000.

The process for treating contaminated glycerol product according to the invention may include a supplementary treatment in which at least one portion of the glycerol obtained at the end of the treatment and at least one portion of the glycerol present in the contaminated glycerol product before the treatment is subjected to at least one separation operation so as to separate the glycerol from the compound containing the alkyl group of the glycerol alkyl ether.

The separation operations may comprise at least one of the group consisting of evaporative concentration, evaporative crystallization, distillation, fractional distillation, stripping and liquid/liquid extraction operations, as described in Application WO 2007/144335, in the name of Solvay S A, from page 6, line 23 to page 8, line 31, the content of which is incorporated herein by reference.

The halo-de-alkoxylation treatment and the separation operation may be carried out consecutively or simultaneously. The term “consecutively” is understood to mean situations during which no operation for separation of the compound containing the alkyl group of the glycerol alkyl ether from the treated glycerol is carried out during the halo-de-alkoxylation treatment. The term “simultaneously” is understood to mean all other situations. The halo-de-alkoxylation treatment and the separation operation are often carried out simultaneously.

At the end of the treatment of contaminated glycerol product according to the invention, the degree of conversion of the glycerol alkyl ethers is generally greater than or equal to 5 mol %, usually greater than or equal to 10 mol %, in many cases greater than or equal to 20 mol %, often greater than or equal to 50 mol %, frequently greater than or equal to 70 mol %, in a lot of cases greater than or equal to 85 mol %, in particular greater than or equal to 90 mol %, specifically greater than or equal to 95 mol % and more particularly greater than or equal to 99.5 mol %. This degree of conversion is generally less than or equal to 99.9 mol %. The degree of conversion is defined as the ratio of the number of moles of glycerol alkyl ethers converted at the end of the treatment to the number of moles of glycerol alkyl ethers submitted to the treatment.

At the end of the treatment of contaminated glycerol product according to the invention, the degree of conversion of the glycerol alkyl ethers to glycerol and/or to glycerol halohydrin (degree of halo-de-alkoxylation) is generally greater than or equal to 5 mol %, usually greater than or equal to 10 mol %, in many cases greater than or equal to 20 mol %, often greater than or equal to 50 mol %, frequently greater than or equal to 70 mol %, in a lot of cases greater than or equal to 85 mol %, in particular greater than or equal to 90 mol %, specifically greater than or equal to 95 mol % and more particularly greater than or equal to 99.5 mol %. This degree of conversion is generally less than or equal to 99.9 mol %.

At the end of the treatment of contaminated glycerol product according to the invention, the degree of conversion of the glycerol alkyl ethers to halogenation products of the glycerol alkyl ethers is generally less than or equal to 80 mol %, often less than or equal to 50 mol %, frequently less than or equal to 30 mol %, in a lot of cases less than or equal to 15 mol %, in particular less than or equal to 10 mol %, specifically less than or equal to 5 mol % and very particularly less than or equal to 0.5 mol %. This degree of conversion is generally greater than or equal to 0.1 mol %.

At the end of the treatment of contaminated glycerol product according to the invention, the degree of conversion of the glycerol present in the contaminated glycerol product before the treatment is generally greater than or equal to 0.01 mol %, often greater than or equal to 0.1 mol %, frequently greater than or equal to 0.5 mol %, in a lot of cases greater than or equal to 1 mol % and in particular greater than or equal to 2 mol %. This degree of conversion is generally lower than or equal to 50 mol %, often lower than or equal to 40 mol %, frequently lower than or equal to 30 mol %, in a lot of cases lower than or equal to 20 mol % and in particular lower than or equal to 10 mol %. This degree of conversion is defined as the ratio of the number of moles of glycerol present in the contaminated glycerol product before the treatment and which have been converted at the end of the treatment to the number of moles of glycerol present in the contaminated glycerol product before the treatment.

The invention also relates to a process for manufacturing a glycerol chlorohydrin comprising:
a) a first step of treating a glycerol product contaminated with at least one glycerol alkyl ether in order to convert at least one portion of the glycerol alkyl ether to glycerol and to at least one compound that contains the alkyl group of the glycerol alkyl ether and that is neither a glycerol alkyl ether nor an alkyl ether of a glycerol halohydrin, in which the glycerol is subjected to a reaction with at least one halo-de-alkoxylation agent, and in which the molar ratio of the total amount of the halo-de-alkoxylation agent introduced during the treatment to the total amount of the glycerol alkyl ether present in the glycerol product before the treatment, is greater than or equal to 0.1 and less than or equal to 1 000 000;
b) optionally, a second step in which at least one portion of the glycerol present at the end of step a) is subjected to at least one separation operation so as to separate the glycerol from the compound(s) containing the alkyl group of the glycerol alkyl ether; and
c) a third step in which at least one portion of the treated glycerol product obtained at the end of step a) and/or at least one portion of the separated glycerol obtained at the end of step b) is subjected to a reaction with hydrogen chloride so as to convert at least one portion of the glycerol to glycerol chlorohydrin.

Step a) of the process for manufacturing glycerol chlorohydrin may be carried out under the conditions described above for the process for treating glycerol product contaminated with at least one glycerol alkyl ether.

Step b) of the process for manufacturing glycerol chlorohydrin may be carried out under the conditions described above for the separation treatment between the glycerol and the compounds containing the alkyl group of the glycerol alkyl ether.

In a preferred embodiment of the process for manufacturing a glycerol chlorohydrin according to the invention, the halo-de-alkoxylation agent used in step a) comprises hydrogen chloride, preferably gaseous hydrogen chloride as described above. The halo-de-alkoxylation agent used in step a) more preferably consists essentially of hydrogen chloride, yet more preferably of gaseous hydrogen chloride as described above.

In the process for treating contaminated glycerol product and in the process for manufacturing glycerol chlorohydrin according to the invention, the contaminated glycerol product may be obtained starting from fossil raw materials and/or renewable raw materials, preferably starting from renewable raw materials, as described in Application WO 2005/054167 by Solvay S A, of which the content, and more specifically the passage from page 1, line 26 to page 4, line 2, is incorporated herein by reference, as described in Application WO 2006/100312 by Solvay S A, of which the content, and more specifically the passage from page 3, line 29 to page 5, line 24, is incorporated herein by reference, and as described in Application WO 2009/000773 in the name of Solvay S A, of which the content, and more specifically the passages on page 10, lines 16 to 23 and on page 11, lines 4 to 25, is incorporated herein by reference.

In the process for treating contaminated glycerol product and in the process for manufacturing glycerol chlorohydrin according to the invention, the contaminated glycerol product may have a content of alkali and/or alkaline-earth metals as described in Application WO 2006/100315 by Solvay S A, of which the content, and more specifically the passage from page 7, line 11 to page 9, line 10, is incorporated herein by reference.

In the process for treating contaminated glycerol product and in the process for manufacturing glycerol chlorohydrin according to the invention, the contaminated glycerol product may contain elements other than the alkali and alkaline-earth metals as described in Application WO 2006/100319 by Solvay S A, of which the content, and more specifically the passages on page 2, lines 3 to 8 and from page 6, line 20 to page 9, line 14, is incorporated herein by reference.

In the process for treating contaminated glycerol product and in the process for manufacturing glycerol chlorohydrin according to the invention, the contaminated glycerol product may contain heavy compounds other than glycerol and for which the boiling points under a pressure of 1 bar absolute are at least 15° C. higher than the boiling point of the glycerol chlorohydrin, as described in Application WO 2006/100316 by Solvay S A, of which the content, and more specifically the passage from page 15, line 32 to page 17, line 33, is incorporated herein by reference.

In the process for treating contaminated glycerol product and in the process for manufacturing glycerol chlorohydrin according to the invention, when the halo-de-alkoxylation agent is hydrogen chloride, the hydrogen chloride may originate, at least partially, from processes as described in Application WO 2005/054167 by Solvay S A, of which the content, and more specifically the passage from page 4, line 32 to page 5, line 18, is incorporated herein by reference, from processes as described in Application WO 2006/106153, of which the content, and more specifically the passages from page 2, line 10 to page 3, line 20 and from page 11, line 1 to page 18, line 29 , is incorporated herein by reference, and from processes as described in Application WO 2007/144335 by Solvay S A, of which the content, and more specifically the passage from page 12, line 14 to page 14, line 21, is incorporated herein by reference. The hydrogen chloride may be purified as described in Application PCT/EP2009/061812, of which the content, and more specifically the passage from page 2, line 31 to page 16, line 12, is incorporated herein by reference.

In the process for manufacturing glycerol chlorohydrin according to the invention, step c) may be carried out in a reaction medium as described in Application WO 2006/106154 by Solvay S A, of which the content, and more specifically the passage from page 14, line 15 to page 17, line 10, is incorporated herein by reference.

The process for treating contaminated glycerol product and the process for manufacturing glycerol chlorohydrin according to the invention may be carried out in reactors produced from or covered with materials that are resistant to the chlorinating agent as described in Application WO 2005/054167 by Solvay S A, of which the content, and more specifically the passage on page 6, lines 3 to 33, is incorporated herein by reference, and in equipment produced from or covered with materials that are resistant to the chlorinating agent, as described in Application WO 2006/100317 by Solvay S A, of which the content, and more specifically the passages from page 2, line 29 to page 3, line 7 and from page 23, line 22 to page 27, line 25, is incorporated herein by reference, and as described in Application WO2009/043796 in the name of Solvay S A, of which the content, and more specifically the passage from page 1, line 30 to page 9, line 17, is incorporated herein by reference.

The process for treating contaminated glycerol product and steps a) and c) of the process for manufacturing glycerol chlorohydrin according to the invention may be carried out in the presence of a catalyst as described in Application WO 2005/054167 by Solvay S A, of which the content, and more specifically the passage from page 6, line 24 to page 7, line 35, is incorporated herein by reference, and in Application WO 2006/020234, of which the content, and more specifically the passage from page 12, line 20 to page 18, line 3, is incorporated herein by reference, and in Application WO 2009/077528 in the name of Solvay S A, of which the content, and more specifically the passage on page 10, lines 4 to 6, is incorporated herein by reference.

Step c) of the process for manufacturing the glycerol chlorohydrin according to the invention may be carried out at a catalyst concentration as described in Application WO 2005/054167 by Solvay S A, of which the content, and more specifically the passage on page 8, lines 6 to 15, is incorporated herein by reference.

Step c) of the process for manufacturing the glycerol chlorohydrin according to the invention may be carried out as described in Application WO 2007/054505 by Solvay S A, of which the content, and more specifically the passages on page 1, lines 24 to 31 and from page 2, line 6 to page 6, line 18, are incorporated herein by reference.

The process for treating contaminated glycerol product and steps a) and c) of the process for manufacturing the glycerol chlorohydrin according to the invention may be carried out in the presence of a solvent as described in Application WO 2005/054167 by Solvay S A, of which the content, and more specifically the passage on page 11, lines 12 to 36, is incorporated herein by reference.

Steps a) and c) of the process for manufacturing the glycerol chlorohydrin according to the invention may be carried out in the presence of a liquid phase comprising heavy compounds other than glycerol, as described in Application WO 2006/100316 by Solvay S A, of which the content, and more specifically the passages on page 2, lines 18 to 25 and from page 15, line 32 to page 17, line 33, is incorporated herein by reference.

The process for treating contaminated glycerol product and steps a) and c) of the process for manufacturing the glycerol chlorohydrin according to the invention may be carried out with stirring using a stirring system as described in Application WO2008/145729 by Solvay S A, of which the content, and more specifically the passages from page 1, line 30 to page 2, line 33 and from page 6, line 22 to page 14, line 31, is incorporated herein by reference.

Step c) of the process for manufacturing the glycerol chlorohydrin according to the invention may be carried out in a liquid reaction medium as described in Application WO 2006/106154 in the name of Solvay S A, of which the content, and more specifically the passages from page 1, line 29 to page 2, line 6 and from page 14, line 15 to page 17, line 10, is incorporated herein by reference.

Step c) of the process for manufacturing the glycerol chlorohydrin according to the invention may be carried out in a reactor, the feeding of which is as described in Application WO 2008/107468 in the name of Solvay S A, of which the content, and more specifically the passages from page 1, lines 29 to page 4, line 27 and from page 5, line 34 to page 9, line 17, is incorporated herein by reference.

In the process for manufacturing the glycerol chlorohydrin according to the invention, the separation of the glycerol chlorohydrin and of the other compounds from the reaction medium may be carried out as described in Application WO 2005/054167 in the name of Solvay S A, of which the content, and more specifically the passage from page 12, line 1 to page 17, line 20, is incorporated herein by reference.

In the process for manufacturing the glycerol chlorohydrin according to the invention, the separation of the glycerol chlorohydrin and of the other compounds from the reaction medium may be carried out according to methods as described in Application WO 2006/100312 in the name of Solvay S A, of which the content, and more specifically the passages on page 2, lines 3 to 10 and from page 20, line 28 to page 28, line 20, is incorporated herein by reference.

In the process for manufacturing the glycerol chlorohydrin according to the invention, the separation of the glycerol chlorohydrin and of the other compounds from the reaction medium may be carried out according to methods as described in Application WO 2006/100313 in the name of Solvay S A, of which the content, and more specifically the passages on page 2, lines 1 to 23 and from page 21, line 7 to page 25, line 25, is incorporated herein by reference.

In the process for manufacturing the glycerol chlorohydrin according to the invention, the separation of the glycerol chlorohydrin and of the other compounds from the reaction medium may be carried out according to methods as described in Application WO 2006/100314 in the name of Solvay S A, of which the content, and more specifically the passages from page 2, line 6 to page 3, line 4 and from page 18, line 33 to page 22, line 29, is incorporated herein by reference.

In the process for manufacturing the glycerol chlorohydrin according to the invention, the separation of the glycerol chlorohydrin and of the other compounds from the reaction medium may be carried out according to methods as described in Application WO 2006/100320 in the name of Solvay S A, of which the content, and more specifically the passages from page 1, line 30 to page 2, line 23 and from page 6, line 25 to page 10, line 28, is incorporated herein by reference.

In the process for manufacturing the glycerol chlorohydrin according to the invention, the separation of the glycerol chlorohydrin and of the other compounds from the reaction medium may be carried out according to methods as described in Application WO 2006/100315 in the name of Solvay S A, of which the content, and more specifically the passages on page 2, lines 3 to 29 and from page 23, line 3 to page 24, line 13, is incorporated herein by reference.

In the process for manufacturing the glycerol chlorohydrin according to the invention, the separation of the glycerol chlorohydrin and of the other compounds from the reaction medium may be carried out according to methods as described in Application WO 2008/110588 in the name of Solvay S A, of which the content, and more specifically the passage from page 1, line 31 to page 27, line 25, is incorporated herein by reference.

In the process for manufacturing the glycerol chlorohydrin according to the invention, when the glycerol chlorohydrin is dichloropropanol, the dichloropropanol is generally obtained in the form of a mixture of the isomers 1,3-dichloropropan-2-ol and 2,3-dichloropropan-1-ol, as described in Application WO 2006/100319 in the name of Solvay S A, of which the content, and more specifically the passage from page 23, line 34 to page 24, line 29, is incorporated herein by reference.

In the process for manufacturing the glycerol chlorohydrin according to the invention, the glycerol chlorohydrin may contain halogenated ketones as described in Application WO 2006/100311 in the name of Solvay S A, of which the content, and more specifically the passages on page 2, lines 22 to 34 and from page 22, line 8 to page 23, line 35, is incorporated herein by reference.

In the process for manufacturing the glycerol chlorohydrin according to the invention, the water which might have been in contact with the equipment walls may be treated as described in Application PCT/EP2009/061546 , of which the content, and more specifically the passage from page 1, line 14 to page 28, line 17, is incorporated herein by reference.

Steps a) and b) of the process for manufacturing a glycerol chlorohydrin according to the invention may be carried out consecutively or simultaneously.

Steps a) and c) of the process for manufacturing a glycerol chlorohydrin according to the invention may be carried out consecutively or simultaneously.

Steps a), b) and c) of the process for manufacturing a glycerol chlorohydrin according to the invention may be carried out consecutively or simultaneously. The term “consecutively” is understood to mean situations where the proportion of glycerol present in the contaminated glycerol product before the treatment which is converted to glycerol chlorohydrin during step a) is less than or equal to 1 mol %. The term “simultaneously” is understood to mean situations where the proportion of glycerol present in the contaminated glycerol product before the treatment which is converted to glycerol chlorohydrin during the glycerol purification process is greater than 1 mol %. Steps a) and c) of the process for manufacturing a glycerol chlorohydrin according to the invention are often carried out simultaneously. Steps a), b) and c) of the process for manufacturing a glycerol chlorohydrin according to the invention are frequently carried out consecutively or simultaneously.

In one particular embodiment of the process for manufacturing glycerol chlorohydrin according to the invention, the halo-de-alkoxylation agent comprises hydrogen chloride, the process includes step b), and steps a), b) and c) are carried out simultaneously.

In another particular embodiment of the process for manufacturing glycerol chlorohydrin according to the invention, the halo-de-alkoxylation agent comprises hydrogen chloride, the process does not include step b), and steps a) and c) are carried out simultaneously.

In those embodiments, the halo-de-alkoxylation agent preferably comprises gaseous hydrogen chloride and more preferably consists essentially of gaseous hydrogen chloride.

The glycerol chlorohydrin may be chosen from the group consisting of monochloropropanediol, dichloropropanol, and any mixture thereof.

The monochloropropanediol may be chosen from the group consisting of 3-chloro-1,2-propanediol, 2-chloro-1,3-propanediol, and any mixture thereof.

The dichloropropanol may be chosen from the group consisting of 1,3-dichloropropan-2-ol, 2,3-dichloropropan-1-ol, and any mixture thereof.

Dichloropropanol is one glycerol chlorohydrin that is very suitable.

The invention also relates to a process for manufacturing an epoxide comprising the process for manufacturing the glycerol chlorohydrin according to the invention, and in which the glycerol chlorohydrin thus obtained is subjected to a dehydro chlorination reaction.

The epoxide may be chosen from the group consisting of glycidol, epichlorohydrin and any mixture thereof. Epichlorohydrin is very suitable.

The process for manufacturing an epoxide according to the invention may be as described in Application WO 2005/054167, in the name of Solvay S A, of which the content, and more specifically the passage from page 19, line 22 to page 22, line 30, is incorporated herein by reference, as described in Application WO 2006/100311, in the name of Solvay S A, of which the content, and more specifically the passages on page 2, lines 22 to 25, and from page 22, line 28 to page 23, line 35, is incorporated herein by reference, as described in Application WO 2008/101866, in the name of Solvay S A, of which the content, and more specifically the passage from page 2, line 1 to page 13, line 16, is incorporated herein by reference, as described in Application WO 2008/152045, in the name of Solvay S A, of which the content, and more specifically the passage from page 9, line 22 to page 13, line 31, is incorporated herein by reference, as described in Application WO 2008/152043 , in the name of Solvay S A, of which the content, and more specifically the passage from page 6, line 16 to page 7, line 22, is incorporated herein by reference, and as described in Application WO 2009/016149, in the name of Solvay S A, of which the content, and more specifically the passage from page 1, line 17 to page 10, line 21, is incorporated herein by reference.

The process for manufacturing the epoxide according to the invention may be integrated into a system for preparing a glycerol chlorohydrin as described in Application WO 2006/106155 in the name of Solvay S A, of which the content, and more specifically the passages on page 2, lines 26 to 31 and from page 22, line 10 to page 23, line 19, is incorporated herein by reference.

The process for manufacturing the epoxide according to the invention may also be carried out as described in Application WO 2006/100318 in the name of Solvay S A, of which the content, and more specifically the passages from page 2, line 23 to page 3, line 26 and from page 24, line 17 to page 31, line 18, is incorporated herein by reference.

The process for manufacturing the epoxide according to the invention may also comprise a step of treating the aqueous effluents as described in Application WO 2009/095429 in the name of Solvay S A, of which the content, and more specifically the passage from page 1, line 24 to page 29, line 27, is incorporated herein by reference.

The invention also relates to a process for manufacturing an epoxy derivative selected from the group consisting of epoxy resins, glycidyl ethers, glycidyl esters, glycidyl amides, glycidyl imides, glycidyl amines, products that can be used as coagulants, wet-strength resins, cationization agents, flame retardants, ingredients for detergents, epichlorohydrin elastomers, halogenated polyethers-polyols, monochloropropanediol and any mixtures of at least two of them, comprising the process for manufacturing the epoxide according to the invention, in which the epoxide is epichlorohydrin, and in which the epichlorohydrin is subjected to a reaction with at least one compound chosen from monoalcohols, monocarboxylic acids, polyols, polyamines, amino alcohols, polyimides, polyamides, polycarboxylic acids, ammonia, amines, polyaminoamides, polyimines, amine salts, phosphoric acid, phosphoric acid salts, phosphorus oxychlorides, phosphoric acid esters, phosphonic acids, esters of phosphonic acids, salts of phosphonic acids, phosphinic acids, esters of phosphinic acids, salts of phosphinic acids, phosphine oxides, phosphines, ethoxylated alcohols, alkylene oxides, and mixtures of at least two of them, or in which the epichlorohydrin according to the invention is subjected to a homopolymerization reaction, or in which epichlorohydrin is subjected to a reaction of oligomerisation, of co-oligomerisation, of condensation, of dehydrochlorination and of hydrolysis, with water, or with a di- or polyhydroxylated compound which may optionally be halogenated and/or have ether oxide bonds and/or double bonds capable of being halogenated in a subsequent stage, or wherein epichlorohydrin is subjected to a reaction with water.

Epichlorohydrin and the uses of epichlorohydrin may be as described in Application WO2008/152045, in the name of Solvay S A, of which the content, and more specifically the passages from page 1, line 18 to page 9, line 2 and from page 31, line 31 to page 63, line 4, is incorporated herein by reference, and as described in Application WO2008/152044, in the name of Solvay S A, of which the content, and more specifically the passages from page 1, line 24 to page 10, line 14 and from page 13, line 3 to page 44, line 8, is incorporated herein by reference.

The examples below are intended to illustrate the invention without, however, limiting it.

### EXAMPLE 1 (ACCORDING TO THE INVENTION)

A glycerol product contaminated with glycerol monomethyl ethers to be treated (150.1 g) has been placed in apparatus composed of a 250 ml, glass, round-bottomed flask, a glass sheath with a thermocouple, a capillary tube for the introduction of gaseous hydrogen chloride (purity: 99.995%), a polytetrafluoroethylene-coated magnetic stirrer bar and a vertical condenser connected to a scrubber fed with an aqueous solution of caustic soda (NaOH). The glycerol has been sparged with gaseous hydrogen chloride, at a flow rate of 1.44 mol/h, with stirring, and by gradually increasing the temperature from 25 to 80° C. over 20 min, under a pressure of 1 bar absolute. The hydrogen chloride flow rate has then been reduced to 0.94 mol/h and the temperature has been maintained at 80° C. The addition of hydrogen chloride has been continued for 4 h 40 min. 117.6 g of hydrogen chloride (4.87 mol) have thus been introduced in total.

The composition of the contaminated glycerol product before sparging with hydrogen chloride is given in Table 1. The glycerol methyl ether content of the contaminated glycerol product before sparging was 11.5 g/kg. The molar ratio of the total amount of hydrogen chloride to the total amount of glycerol methyl ethers introduced during the 5 h of the treatment was 299.

The composition of the liquid phase in the reactor after sparging with hydrogen chloride is given in Table 1. The degree of halo-de-alkoxylation of the glycerol methyl ethers came to 80% overall. The degree of conversion of the glycerol present in the contaminated glycerol product before the treatment is lower than 50% mol.

TABLE 1

| Glycerol components | Before sparging | After sparging |
|---|---|---|
| (g/kg) | with HCl | with HCl |
| HCl | | 118 |
| 2-Methoxy-1,3-propanediol | 2.4 | 0.38 |
| 3-Methoxy-1,2-propanediol | 9.1 | 1.2 |
| 1-Methoxy-3-chloro-2-propanol | 0.02 | 0.32 |
| Glycerol | 968 | 538 |
| 3-Chloro-1,2-propanediol | 0 | 256 |
| 2-Chloro-1,3-propanediol | 0 | 13 |
| 1,3-Dichloro-2-propanol + 2,3- | 0 | 11 |
| dichloro-1-propanol | | |
| Diglycerol | 2.8 | 4.9 |
| Cyclic diglycerol | 0.15 | 0.51 |
| Diglycerol monochlorohydrin | 0 | 1.1 |
| Acetaldehyde | 0.011 | 0.014 |
| Propionaldehyde | 0.009 | n.d. |
| Acrolein | n.d. | <0.005 |
| Butyraldehyde | <0.005 | n.d. |
| Acetone | 0.011 | 0.006 |
| Methanol | 2.5 | 0.062 |
| n-Propanol | 0.016 | n.d. |
| 1,2-Propanediol | 0.14 | 0.043 |
| 1,3-Propanediol | 0.054 | n.d. |
| Palmitic acid | <0.05 | 0.08 |
| Oleic acid | 0.08 | n.d. |
| Linoleic acid | <0.05 | n.d. |
| Stearic acid | 0.08 | 0.14 |
| Methyl palmitate | 0.31 | <0.05 |
| Methyl oleate | 2.8 | 0.36 |
| Methyl linoleate | 0.82 | 0.06 |
| Methyl stearate | 0.05 | n.d. |
| Glyceryl acetate | 0.37 | 0.26 |
| Glyceryl monopalmitate | 0.15 | 0.5 |
| Glyceryl monooleate and | 2 | n.d. |
| monolinoleate | | |
| Glyceryl monostearate | 0.09 | 0.08 |

n.d.: not detected

### EXAMPLE 2 (ACCORDING TO THE INVENTION)

A glycerol product contaminated with glycerol monomethyl ethers to be treated (150.1 g) has been placed in apparatus composed of a 250 ml, glass, round-bottomed flask, a glass sheath with a thermocouple, a capillary tube for the introduction of gaseous hydrogen chloride (purity: 99.995%), a polytetrafluoroethylene-coated magnetic stirrer bar and a vertical condenser cooled with water at room temperature. The outlet of the condenser has been connected to a washing bottle containing 240 g of carbon tetrachloride maintained at 0° C. in order to trap volatile organic compounds evaporated from the reaction mixture. The gaseous effluent from the washing bottle which contained the excess of hydrogen chloride has been neutralized in a scrubber connected to the washing bottle outlet and fed with an aqueous solution of caustic soda (NaOH). The glycerol has been sparged with gaseous hydrogen chloride, at a flow rate of 0.45 mol/h, with stirring, during 106 minutes, at a temperature maintained between 90° C. and 100° C. and under a pressure of 1 bar absolute.

The composition of the contaminated glycerol product before sparging with hydrogen chloride is given in Table 2. The glycerol methyl ether content of the contaminated glycerol product before sparging was 2.6 g/kg. The molar ratio of the total amount of hydrogen chloride to the total amount of glycerol methyl ethers introduced during the 106 min of the treatment was 205.

The composition of the liquid phase in the reactor after sparging with hydrogen chloride appears in Table 2. The degree of halo-de-alkoxylation of the glycerol methyl ethers comes to 86% overall. 0.029 g of monochloromethane have been recovered in the washing bottle. The degree of conversion of the glycerol present in the contaminated glycerol product before the treatment is lower than 50% mol.

TABLE 2

| Glycerol components | Before sparging | After sparging |
|---|---|---|
| (g/kg) | with HCl | with HCl |
| HCl | | 92 |
| 2-Methoxy-1,3-propanediol | 0.2 | 0.1 |
| 3-Methoxy-1,2-propanediol | 2.4 | 0.2 |
| 1-Methoxy-3-chloro-2-propanol | n.d. | n.d. |
| Glycerol | M.C. | M.C. |
| 3-Chloro-1,2-propanediol | 0.06 | 84 |
| 2-Chloro-1,3-propanediol | n.d. | 6.3 |
| 1,3-Dichloro-2-propanol + 2,3- | n.d. | 1.4 |
| dichloro-1-propanol | | |
| Diglycerol | 0.6 | 5.6 |
| Cyclic diglycerol | 0.4 | n.d. |
| Diglycerol monochlorohydrin | n.d. | 0.97 |
| Not identified compounds | 1.9 | 2.5 |

n.d.: not detected;
M.C.: Main Constituent

### EXAMPLE 3 (ACCORDING TO THE INVENTION)

A glycerol product contaminated with glycerol monomethyl ethers to be treated (157.6 g) has been placed in apparatus composed of a 500 ml, glass, round-bottomed flask, a glass sheath with a thermocouple, a tube for the introduction of concentred hydrochloric acid (36.4% by weight of hydrogen chloride), a polytetrafluoroethylene-coated magnetic stirrer bar and a vertical condenser cooled with water at room temperature. The outlet of the condenser has been connected to a scrubber fed with water. The concentred hydrochloric acid has been added at a constant flow rate of 37 ml/h (0.44 mol/h), with stirring, during 106 minutes, at a temperature maintained between 90° C. and 100° C. and under a pressure of 1 bar absolute. The weight of the mixture at the end of the treatment was 233.3 g.

The composition of the contaminated glycerol product before the treatment with hydrochloric acid is given in Table 3. The glycerol methyl ether content of the contaminated glycerol product before the treatment was 2.18 g/kg. The molar ratio of the total amount of hydrogen chloride to the total amount of glycerol methyl ethers introduced during the 106 min of the treatment was 238.

The composition of the liquid phase in the reactor after the treatment with hydrochloric acid appears in Table 3. The degree of halo-de-alkoxylation of the glycerol methyl ethers comes to 8% overall.

TABLE 3

| Glycerol components | Before treatment | After treatment |
|---|---|---|
| (g/kg) | with HCl | with HCl |
| HCl | | 117.3 |
| Water | | 208 |
| 2-Methoxy-1,3-propanediol | 0.08 | 0.06 |
| 3-Methoxy-1,2-propanediol | 2.1 | 1.3 |
| 1-Methoxy-3-chloro-2-propanol | n.d. | n.d. |
| Glycerol | M.C. | M.C. |
| 3-Chloro-1,2-propanediol | n.d. | 1.35 |
| 2-Chloro-1,3-propanediol | n.d. | 0.32 |
| 1,3-Dichloro-2-propanol + 2,3- | n.d. | n.d. |
| dichloro-1-propanol | | |
| Diglycerol | 0.40 | 0.53 |
| Cyclic diglycerol | n.d. | n.d. |
| Diglycerol monochlorohydrin | n.d. | n.d. |
| Not identified compounds | 0.59 | 1.07 |

n.d.: not detected;
M.C.: Main Constituent

1. A process for treating a glycerol product contaminated with at least one glycerol alkyl ether in order to convert at least one portion of the glycerol alkyl ether to glycerol, in which the glycerol product is subjected to a reaction with at least one halo-de-alkoxylation agent, and in which the molar ratio of the total amount of the halo-de-alkoxylation agent which is introduced during the treatment to the total amount of the glycerol alkyl ether present in the glycerol product before the treatment, is greater than or equal to 0.1 and less than or equal to 1 000 000.

2. The process according to claim 1, in which, in addition, at least one compound is formed that contains the alkyl group of the glycerol alkyl ether and that is neither a glycerol alkyl ether nor an alkyl ether of a glycerol halohydrin.

3. The process according to claim 1, wherein the glycerol alkyl ether is chosen from the group consisting of glycerol alkyl monoethers, glycerol alkyl diethers, glycerol alkyl triethers, and any mixture of at least two of them, and in which the alkyl groups of the glycerol alkyl ethers are selected independently from the group consisting of methyl, ethyl, propyl, butyl, pentyl, hexyl, heptyl, octyl groups, and any combination of at least two of them.

4. The process according to claim 1, wherein the content of alkyl ether in the glycerol product before the treatment is greater than or equal to 0.001 g per kg of contaminated glycerol product and less than or equal to 100 g per kg of contaminated glycerol product.

5. The process according to claim 1, wherein the halo-de-alkoxylation agent contains at least one hydrogen halide.

6. The process according to claim 5, wherein the hydrogen halide contains hydrogen chloride.

7. The process according to claim 6, wherein the hydrogen chloride is used in the form of an aqueous solution, and wherein the amount of hydrogen chloride relative to the sum of the amounts of hydrogen chloride and of water is higher than or equal to 40% by weight.

8. The process according to claim 6, wherein the hydrogen chloride is used in gas form optionally in admixture with at least one other gaseous compound selected from the group consisting of nitrogen, oxygen, carbon dioxide, steam, a noble gas, and any mixture of at least two of them.

9. The process according to claim 8, wherein the hydrogen chloride in the gas mixture is higher than or equal to 99% mol.

10. The process according to claim 2, wherein the compound containing the alkyl group of the glycerol alkyl ether is an alkyl halide.

11. The process according to claim 1, being carried out in batch or continuous mode, wherein the reaction is performed under at least one of the following conditions:
at a temperature greater than or equal to 20° C. and less than or equal to 160° C.;
at a pressure greater than or equal to 0.3 bar absolute and less than or equal to 100 bar absolute;
for a time greater than or equal to 1 h and less than or equal to 100 h when the process is carried out in batch mode, or for a residence time greater than or equal to 1 h and less than or equal to 100 h when the process is carried out in continuous mode.

12. A process for manufacturing an epoxide, comprising:
a) a first step of treating a glycerol product contaminated with at least one glycerol alkyl ether according to the process of claim 1;
b) optionally, a second step in which at least one portion of the glycerol present at the end of step a) is subjected to at least one separation operation so as to separate the glycerol from the compound(s) containing the alkyl group of the glycerol alkyl ether;
c) a third step in which at least one portion of the treated glycerol product obtained at the end of step a) and/or at least one portion of the separated glycerol obtained at the end of step b) is subjected to a reaction with hydrogen chloride so as to convert at least one portion of the glycerol to glycerol chlorohydrin,
in which the glycerol chlorohydrin thus obtained is subjected to a dehydrochlorination reaction.

13. The process according to claim 12, wherein the halo-de-alkoxylation agent used in step a) and the hydrogen chloride used at step c) are gaseous hydrogen chloride.

14. (canceled)

15. A process for manufacturing an epoxy derivative selected from the group consisting of epoxy resins, glycidyl ethers, glycidyl esters, glycidyl amides, glycidyl imides, glycidyl amines, products that can be used as coagulants, wet-strength resins, cationization agents, flame retardants, ingredients for detergents, epichlorohydrin elastomers, halogenated polyethers-polyols, monochloropropanediol and any mixtures of at least two of them, comprising carrying out the process according to claim 13, wherein the epoxide is epichlorohydrin and wherein the epichlorohydrin is subjected to a reaction with at least one compound selected from the group consisting of monoalcohols, monocarboxylic acids, polyols, polyamines, amino alcohols, polyimides, polyamides, polycarboxylic acids, ammonia, amines, polyaminoamides, polyimines, amine salts, phosphoric acid, phosphoric acid salts, phosphorus oxychlorides, phosphoric acid esters, phosphonic acids, esters of phosphonic acids, salts of phosphonic acids, phosphinic acids, esters of phosphinic acids, salts of phosphinic acids, phosphine oxides, phosphines, ethoxylated alcohols, alkylene or phenylene oxides, and mixtures of at least two of them, or wherein epichlorohydrin is subjected to a homopolymerization reaction, or wherein epichlorohydrin is subjected to a reaction selected from the group consisting of oligomerisation, co-oligomerisation, condensation, dehydrochlorination, and hydrolysis, with water, or with a di- or polyhydroxylated compound which may optionally be halogenated and/or have ether oxide bonds and/or double bonds capable of being halogenated in a subsequent stage, or wherein epichlorohydrin is subjected to a reaction with water.

16. The process according to claim 12, wherein the epoxide is epichlorohydrin, and wherein the glycerol chlorohydrin is dichloropropanol.
